(19) United States
(12) Patent Application Publication
(10) Pub. No.: US 2022/0318919 A1
(43) Pub. Date: Oct. 6, 2022

(54) SUBROGATION COLLECTION SYSTEM AND METHOD

(71) Applicant: ZAF (Zero Attorney Fees), Nuttal Brown & Coutts, Cottonwood Heights, UT (US)

(72) Inventors: Scott Brown, Sandy, UT (US); Tyler Brown, Sandy, UT (US); Brian Coutts, Centerville, UT (US); Eric Eliason, Sandy, UT (US); Nate Obray, Logan, UT (US)

(21) Appl. No.: 17/712,866

(22) Filed: Apr. 4, 2022

Related U.S. Application Data
(60) Provisional application No. 63/171,273, filed on Apr. 6, 2021.

(51) Int. Cl. G06Q 40/08; G06Q 20/30; G16H 10/60; G06Q 50/18; G06F 16/14; G06Q 20/32

(52) U.S. Cl. CPC G06Q 40/08; G06Q 20/308; G16H 10/60; G06Q 50/18; G06F 16/14; G06Q 20/322

(57) ABSTRACT

A computerized method for collecting subrogation in a personal injury case, comprising using a computer network that includes servers, processors, databases, and software to facilitate the establishment of a contractual relationship between a health plan provider and a legal entity acting as a subrogation collection entity; and using the computer network to facilitate the establishment of a contractual relationship between the health plan provider and a constituent of a health plan offered by the health plan provider, wherein the health plan includes access by the constituent of the health plan to personal injury legal services offered by the legal entity acting as a subrogation collection entity at no cost or reduced low cost to the constituent of the health plan.

### CROSS-REFERENCE TO RELATED APPLICATIONS

This patent application claims the benefit of U.S. Provisional Patent Application Ser. No. 63/171,273 filed on Apr. 6, 2021 and entitled “Subrogation Collection System and Method,” the disclosure of which is hereby incorporated by reference herein in its entirety and made part of the present U.S. utility patent application for all purposes.

### BACKGROUND

The disclosed inventive subject matter relates in general to computerized systems and methods for simultaneously processing and managing personal injury cases and insurance provider subrogation claims, and more specifically to an automated system for allowing insurers that offer and provide health plans to collect subrogation to which they are legally entitled without damaging the professional and working relationships these entities have with their health plan constituents (i.e., policy holders).

The disclosed system, referred to herein as ZAF Subrogation, the ZAF Subrogation System, or simply ZAF, provides a method of legal practice that involves the establishment of unique relationships and the management thereof with various automated processes. ZAF Subrogation simultaneously addresses and serves the needs of two industries: personal injury legal practice and subrogation recovery services.

“Subrogation” is defined as reimbursement for accident-related medical expenses that a health plan provider (referred to herein as “health plan”) pays on behalf of its customers (i.e., “members” or “constituents”) who have obtained a recovery in connection with a personal injury case. Historically, health plans have struggled with collecting the subrogation to which they are entitled from personal injury settlements. If there is no personal injury recovery, there is no subrogation right. When a health plan is processing medical bills, there are few indicators that allow the health plan to know when a particular medical visit is connected to a personal injury case. If the health plan fails to assert its subrogation right prior to a member settling the personal injury case, the health plan typically fails to receive any subrogation.

Although health plans have robust subrogation rights in most jurisdictions, there are many practical challenges to enforcing these rights. Health plans depend on their members to report the existence of a personal injury claim. Members, however, do not typically wish to pay subrogation back to health plans and tend to be less than forthcoming with relevant case information. This creates an information gap and often results in missed subrogation.

In cases where subrogation is asserted by a health plan, the relationship between members and their health plan is harmed for obvious reasons. From the member's perspective, the health plan is just taking the member's settlement money away, even though the member has paid the premiums required by the health plan over the life of a particular policy. Members are almost always caught off guard when they learn that subrogation rights even exist. From the health plan's perspective, the law is clearly in its favor and it is entitled to reimbursement. In view of this conflict, the two sides consistently battle over subrogation. Lawyers retained by members help their clients negotiate discounted subrogation amounts. Health plans generally agree to at least a one-third discount on the amount they are owed.

Another reason a health plan may fail to collect subrogation is the reluctance of its members to assert personal injury claims against third parties. If a member forgoes his or her personal injury claim, the health plan does not have the opportunity to collect subrogation. Accordingly, the most effective way for a health plan provider to collect the subrogation to which it is entitled is for the health plan provider to partner with and pay for the services of a legal entity such as a lawyer or law firm that handles and settles the personal injury cases of the members of the health plan.

Accordingly, the primary challenges associated with collecting subrogation include: (i) identifying the existence of a personal injury case and asserting subrogation; (ii) maintaining a positive and constructive relationship between a health plan and its members; (iii) collecting full subrogation when a one-third discount is recognized as the industry standard; and (iv) convincing members to pursue, rather than forgo their personal injury cases. Therefore, there is an ongoing need for a system and method for allowing health plans to collect the subrogation to which they are legally entitled without damaging the professional and working relationships these health plans have with their members.

### SUMMARY

The following provides a summary of certain example implementations of the disclosed inventive subject matter. This summary is not an extensive overview and is not intended to identify key or critical aspects or elements of the disclosed inventive subject matter or to delineate its scope. However, it is to be understood that the use of indefinite articles in the language used to describe and claim the disclosed inventive subject matter is not intended in any way to limit the described inventive subject matter. Rather the use of “a” or “an” should be interpreted to mean “at least one” or “one or more”.

One implementation of the disclosed technology provides a system, which may be partially or entirely computerized, for collecting subrogation from personal injury settlements, comprising a computer network that includes servers, processors, databases, and software; wherein the computer network is configured to facilitate establishment of a contractual relationship between a health plan provider and a legal entity acting as a subrogation collection entity; wherein the computer network is configured to facilitate establishment of a contractual relationship between the health plan provider and a constituent (e.g., member or purchaser) of a health plan offered by the health plan provider, wherein the health plan includes access by the constituent of the health plan to personal injury legal services offered by the legal entity acting as a subrogation collection entity at either no cost or reduced cost to the constituent of the health plan; wherein the computer network is configured to access information stored in one or more databases that includes electronic health information about the constituent of the health plan; wherein the stored information includes medical diagnosis and procedure codes indicative of an accident in which the constituent of the health plan has been involved that results in a personal injury case is periodically reviewed; wherein the computer network is configured to contact the constituent of the health plan and gains approval from the constituent of the health plan to utilize the legal services of the subrogation collection entity; wherein the computer network is configured to create a subrogation case file for the health plan upon receiving approval from the constituent to utilize the personal injury legal services of the legal entity acting as a subrogation collection entity; wherein the computer network is configured to retrieve information from the databases that includes medical records of the constituent of the health plan regarding accident-related medical expenses paid to the constituent of the health plan by the health plan provider and use the accident-related medical expenses to calculate total subrogation payable to the health plan provider; wherein the computer network is configured to obtain approval from the constituent of the health plan to facilitate personal injury legal services and settle the personal injury case of the constituent of the health plan at no cost or reduced cost to the constituent of the health plan; and wherein the computer network is configured to facilitate payment of up to 100% of the calculated subrogation by the subrogation collection entity to the health plan provider.

The system may charge a predetermined fee to the health plan provider for software and services of the legal entity acting as a subrogation collection entity. The legal entity acting as a subrogation collection entity may have access to the databases and servers of a personal injury lawyer or personal injury law firm and may actually be a single entity providing both subrogation collection services to health plans and personal injury services to constituents of the health plan operating a shared database for both purposes. The system may be entirely automated or predetermined aspects of the system may be automated (i.e., the system may be partially automated). The computer network may be accessed using application software resident on hand-held devices and the hand-held devices may include mobile telephones, tablet computers, or other similar devices. Additionally, the network may be accessed by way of browser-based web applications accessible on any device having an internet connection.

Another implementation of the disclosed technology provides a computerized method for collecting subrogation in a personal injury case, comprising using a computer network that includes servers, processors, databases, and software to facilitate the establishment of a contractual relationship between a health plan provider and a legal entity acting as a subrogation collection entity; using the computer network to facilitate the establishment of a contractual relationship between the health plan provider and a constituent (e.g., member or purchaser) of a health plan offered by the health plan provider, wherein the health plan includes access by the constituent of the health plan to personal injury legal services offered by the legal entity acting as a subrogation collection entity at no cost or reduced low cost to the constituent of the health plan; using the computer network to access information stored in one or more databases that includes electronic health information about the constituent of the health plan; periodically reviewing the stored information includes medical diagnosis and procedure codes indicative of an accident in which the constituent of the health plan has been involved, wherein the accident results in a personal injury case; using the computer network to contact the constituent of the health plan and gain approval from the constituent of the health plan to utilize the legal services of the subrogation collection entity; using the computer network to create a subrogation case file for the health plan upon receiving approval from the constituent to utilize the personal injury legal services of the legal entity acting as a subrogation collection entity; using the computer network to retrieve information from the databases that includes medical records of the constituent of the health plan regarding accident-related medical expenses paid to the constituent of the health plan by the health plan provider and using the accident-related medical expenses to calculate total subrogation payable to the health plan provider; using the computer network to obtain approval from the constituent of the health plan to settle the personal injury case of the constituent of the health plan at no cost or reduced cost to the constituent of the health plan; and using the computer network to facilitate payment of up to 100% of the calculated subrogation by the subrogation collection entity to the health plan provider.

The method may further comprise charging a predetermined fee to the health plan provider for access to the software and services of the legal entity acting as a subrogation collection entity. The legal entity acting as a subrogation collection entity may have access to the databases and servers of a personal injury lawyer or personal injury law firm. The method may be entirely automated or certain predetermined aspects of the method may automated while others are manual. The computer network may be accessed using application software resident on hand-held devices, and wherein the hand-held devices may include mobile telephones, tablet computers, and the like. Additionally, the network may be accessed by way of browser-based web applications accessible on any device having an internet connection.

Still another implementation of the disclosed technology provides an automated method for collecting subrogation in a personal injury case, comprising providing a computer network, wherein the computer network includes servers, processors, databases, and software; using the computer network to facilitate the establishment of a contractual relationship between a health plan provider and a legal entity acting as a subrogation collection entity; using the computer network to facilitate the establishment of a contractual relationship between the health plan provider and a constituent (e.g., member or purchaser) of a health plan offered by the health plan provider, wherein the health plan includes access by the constituent of the health plan to personal injury legal services offered by the legal entity acting as a subrogation collection entity at no cost or reduced low cost to the constituent of the health plan; using the computer network to access information stored in one or more databases that includes electronic health information about the constituent of the health plan; periodically reviewing the stored information includes medical diagnosis and procedure codes indicative of an accident in which the constituent of the health plan has been involved, wherein the accident results in a personal injury case; using the computer network to contact the constituent of the health plan and gain approval from the constituent of the health plan to utilize the legal services of the subrogation collection entity; using the computer network to create a subrogation case file for the health plan upon receiving approval from the constituent to utilize the personal injury legal services of the legal entity acting as a subrogation collection entity; using the computer network to retrieve information from the databases that includes medical records of the constituent of the health plan regarding accident-related medical expenses paid to the constituent of the health plan by the health plan provider and using the accident-related medical expenses to calculate total subrogation payable to the health plan provider; using the computer network to obtain approval from the constituent of the health plan to settle the personal injury case of the constituent of the health plan at no cost or reduced cost to the constituent of the health plan; using the computer network to facilitate payment of up to 100% of the calculated subrogation by the subrogation collection entity to the health plan provider; and charging a predetermined fee to the health plan provider for the software and services of the legal entity acting as a subrogation collection entity.

The legal entity acting as a subrogation collection entity may have access to the databases and servers of a personal injury lawyer or personal injury law firm. The computer network may be remotely accessed using application software resident on hand-held devices. The hand-held devices may include mobile telephones, tablet computers, and the like. Additionally, the network may be accessed by way of browser-based web applications accessible on any device having an internet connection.

It should be appreciated that all combinations of the foregoing concepts and additional concepts discussed in greater detail below (provided such concepts are not mutually inconsistent) are contemplated as being part of the inventive subject matter disclosed herein and may be implemented to achieve the benefits as described herein. Additional features and aspects of the disclosed system, devices, and methods will become apparent to those of ordinary skill in the art upon reading and understanding the following detailed description of the example implementations. As will be appreciated by the skilled artisan, further implementations are possible without departing from the scope and spirit of what is disclosed herein. Accordingly, the drawings and associated descriptions are to be regarded as illustrative and not restrictive in nature.

### BRIEF DESCRIPTION OF THE DRAWINGS

The accompanying drawings, which are incorporated into and form a part of the specification, schematically illustrate one or more example implementations of the disclosed inventive subject matter and, together with the general description given above and detailed description given below, serve to explain the principles of the disclosed subject matter, and wherein:

FIG. 1 provides a flowchart depicting an example implementation of the disclosed system and method for collecting subrogation in a personal injury case, wherein the various steps in the method are automatically executed by one or more servers, wherein one or more of the servers includes at least one processor for running subrogation collection software;

FIGS. 2A-2B provide a flowchart depicting an example implementation of the disclosed subrogation system and method wherein a series of queries executed by the subrogation collection software is shown in chronological order; and

FIGS. 3A-3C provide a system schematic depicting another example implementation of the disclosed system and method for collecting subrogation in a personal injury case, wherein the various steps in the method are automatically executed by one or more servers communicating with each other using a web-based system, wherein one or more of the servers includes at least one processor for running subrogation collection software.

### DETAILED DESCRIPTION

Example implementations are now described with reference to the Figures. Reference numerals are used throughout the detailed description to refer to the various elements and structures. Although the following detailed description contains many specifics for the purposes of illustration, a person of ordinary skill in the art will appreciate that many variations and alterations to the following details are within the scope of the disclosed inventive subject matter. Accordingly, the following implementations are set forth without any loss of generality to, and without imposing limitations upon, the claimed subject matter.

Health plans spend significant amounts of money pursuing subrogation, and most health plans ultimately hire a subrogation recovery vendor to achieve their goals. Although these vendors are specialists, they face the same previously-described problems encountered by health plans. However, the disclosed systems and methods effectively overcome these issues. Rather than pursuing new and better ways to fight with members and personal injury attorneys to recover more subrogation, the ZAF Subrogation Model eliminates the conflict entirely and collects substantially more subrogation. Unlike typical subrogation vendors, ZAF's unique approach solves inherent problems and challenges associated with subrogation collection and does so while simultaneously providing a significant legal benefit to the members of a participating health plan.

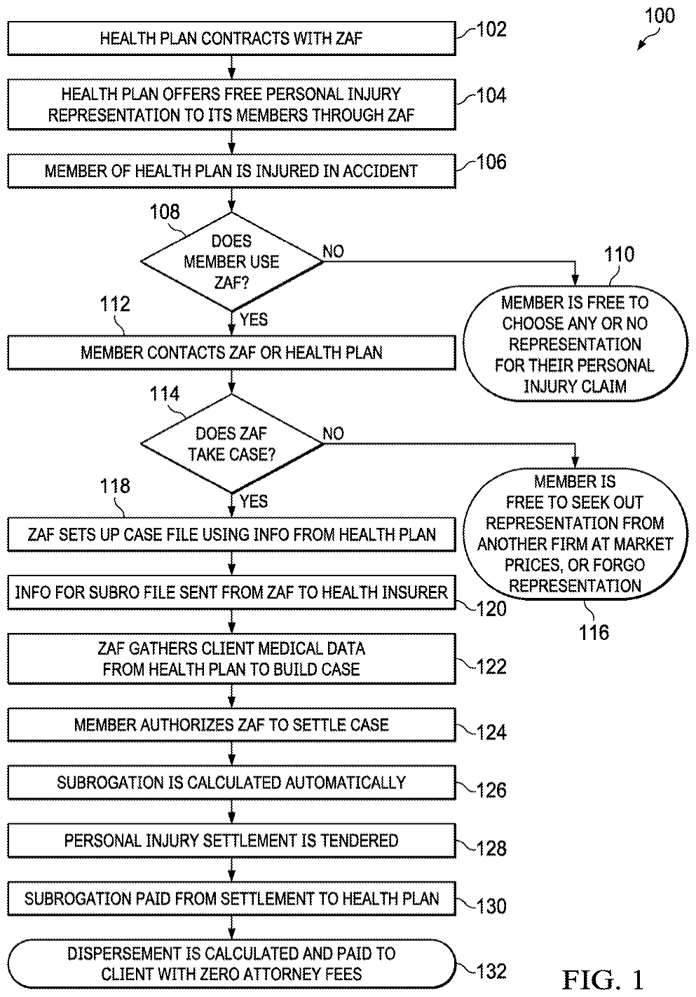

With reference generally to FIG. 1, the disclosed ZAF Subrogation System interfaces with the systems of health plans to offer “zero attorney fee” personal injury legal representation, or discounted personal injury legal representation, to all or some of the members of the health plan. As a virtual (or actual, in some implementations) legal entity handling a personal injury case, the ZAF Subrogation System ensures that the health plan recovers up to 100% subrogation from every personal injury settlement. The additional amount of subrogation generated by the ZAF Subrogation System typically offsets some or all of the cost incurred by the health plan by using the system.

The ZAF subrogation system receives payment directly from the system used by a health plan for the service provided. The members of a health plan are informed in advance regarding the availability of the benefits offered by the ZAF system. Members are encouraged to directly access either the ZAF Subrogation System or the health plan system if the member is involved in an accident. When a member accesses the ZAF Subrogation System, the system evaluates the merits of the case based on data received from the member. If evaluated data meets certain predetermined criteria and the case appears to be viable, the ZAF Subrogation System automatically initiates formal representation of the member. If the data does not meet certain predetermined criteria, the ZAF Subrogation System will indicate refusal of representation to the member.

For members who choose not to access the ZAF Subrogation System after an accident, or simply neglect to do so, the ZAF Subrogation System will automatically identify and contact those members. Health plans maintain databases containing electronic health information. Such databases include but are not limited to diagnostic and procedure codes. Computer systems operated by health plans scan such data for highly suspect diagnoses; for example, the type of diagnoses typically associated with automobile accidents. These highly suspect diagnoses are transmitted by the health plan to the ZAF Subrogation System with the contact information of the relevant member. These members are then contacted through automated text and e-mail messages or other means.

Once an initially non-responsive member has been successfully contacted, their case is evaluated against predetermined criteria. If the case is considered viable, representation of the member is undertaken by the ZAF Subrogation System. The ZAF Subrogation System is configured to periodically provide health plans with updated lists of accepted cases. This list includes the information required by the health plan for creating an electronic subrogation file for each member that has been injured and that has initiated a personal injury case. The ZAF Subrogation System also generates a unique case identifier, which is transmitted to and stored by the health plan. As each case progresses, the ZAF Subrogation System automatically retrieves medical bill data and other relevant records from the databases maintained by health plans. This data is critical when presenting the personal injury case to the bodily injury insurer. Normally, personal injury lawyers must pay medical providers to provide this documentation. Health plans can share it with the ZAF Subrogation System cost-free, thereby saving members money regarding costs that must be advanced, and which are deducted from the eventual personal injury settlement amount.

When a personal injury case settles, the ZAF Subrogation System retrieves data from the health plan databases to calculate the total amount paid by the health plan in accident-related medical expenses. The amount calculated is the total subrogation amount owed to the health plan. The ZAF Subrogation System collects the settlement monies, pays the health plan up to 100% subrogation (policy limits allowing), and disburses the net recovery to the member.

The disclosed technology may be used: (i) to facilitate discounted or zero attorney fee representation for accident victims who have health insurance while increasing their net recovery; (ii) to eliminate the conflict around subrogation by automatically facilitating up to 100% subrogation on every case; (iii) to retrieve data from a health plan's database to assist with handling of a health plan member's personal injury case; and (iv) to expedite subrogation collection and disbursement of the member's personal injury settlement.

More specifically, FIG. 1 provides a process work flow or flowchart depicting an example implementation of the disclosed system and method for collecting subrogation in a personal injury case, wherein the various steps in the method are automatically executed by one or more servers, and wherein one or more of the servers includes at least one processor for running subrogation collection software. With direct reference to FIG. 1, system 100 includes the following process or method steps. A health plan contracts with ZAF at step 102; the health plan offers free or discounted personal injury legal services to its members through ZAF at step 104; a member of the health plan experiences an injury in accident at step 106; and a determination as to whether or not the member uses ZAF is made at step 108. If no, the member chooses any or no representation for their personal injury claim at step 110. If yes, the member contacts ZAF or health plan at step 112. A decision as to whether or not ZAF takes the case is made at step 114. If no, the member either seeks out representation from another firm at market prices, or forgoes representation at step 116. If yes, ZAF sets up a case file using information from the health plan at step 118. Information for a subrogation file is sent from ZAF to health plan at step 120; ZAF gathers client medical data from the health plan to a build case at step 122; the member provides authorization to settle the case at step 124; subrogation is calculated automatically at step 126; a personal injury settlement is tendered at step 128; subrogation is paid from the settlement to the health plan at step 130; and disbursement is calculated and paid to the member at step 132.

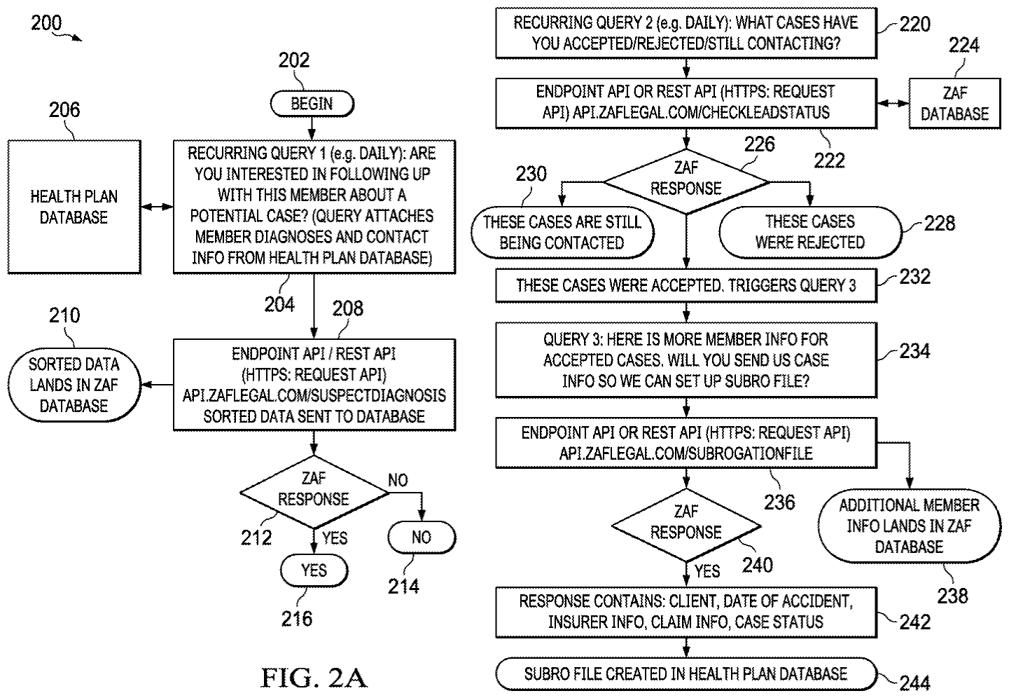

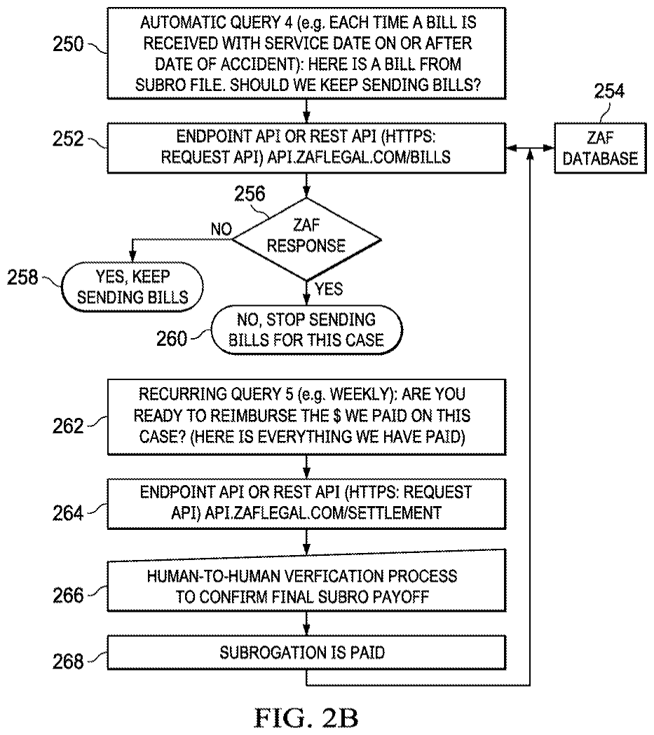

With reference generally to FIGS. 2A-2B, the case intake, and the subsequent subrogation process, is facilitated by automated data exchanges. The central feature of this technology is the ZAF REST Application Programming Interface (API) Endpoint implemented on top of the HTTPS protocol that is queried by a program implemented by the health plan that transfers JSON formatted data from the health plan database, along with certain queries, to the ZAF REST API endpoint. The ZAF REST API endpoint is a webserver running an application to receive JSON requests, process data, extract information, and transfer certain data into a database where that data is stored. The database could be any method used to store data as its own layer in the service (e.g. a Relation Database Service (RDS) Instance run through AWS). The series of queries and associated data flows as detailed below.

Query 1. As the health plan receives new medical bills to process on behalf of its members, the health plan software, or health plan client (hereafter “HPC”), extracts all or some of these bills that are deemed to be “highly suspect diagnoses” and sends them to the ZAF REST API endpoint in a recurring query (submitted at whatever frequency deemed prudent by the health plan, e.g. daily) that asks, “Are you interested in following up with this member about a potential case?” Attached to this query is a copy of the bill, data associated with the bill that has been input into the health plan database, the contact information for the member, and the health plan ID number (in JSON format) for the member.

The ZAF REST API endpoint sorts and prioritizes the data by scanning for highly suspect diagnoses most indicative of traumatic injury. These are potentially accident-related, which is suggestive of a possible personal injury claim. The ZAF REST API endpoint responds to the HPC with a yes/no answer depending on whether the bill was flagged as potentially being accident-related. Some diagnostic codes are highly suggestive of being accident-related, while others merely have the possibility of being accident related. The ZAF REST API endpoint sorts suspect diagnoses based on an algorithm that categorizes the likelihood, from high likelihood to low likelihood, of a particular diagnosis being accident related. The diagnoses that are not accident-related (e.g., congenital illnesses, diseases, etc.) are discarded. All other relevant data is stored in a designated ZAF database. Members of interest are contacted by the ZAF Subrogation System through automated and non-automated processes to investigate the possible existence of a viable personal injury claim. In an example implementation of the ZAF Subrogation System, Query 1, is sent to httpsapi.zaflegal.com/suspect diagnosis.

Query 2. A second recurring query is sent out at a predetermined frequency or interval (e.g., daily) by the HPC. This is a query with no data attached. More specifically, the query is: “What cases have been accepted by ZAF?” The ZAF REST API endpoint receives this request, then retrieves the relevant data (i.e., cases that have been accepted) from the ZAF database. This data is sent back to the HPC as a response. The ZAF REST API endpoint extracts case information from the ZAF database, including the name of the member, the member health plan ID number, date of accident, description of accident, primary injuries, bodily injury insurer, bodily injury adjuster name/phone/fax/address, claim number, and policy limits if known. The HPC populates a subrogation file in the health plan database using the data contained in the ZAF response. In an example implementation of the ZAF Subrogation System, Query 2 is sent to api.zaflegal.com/subrogationfile.

Query 3. Query 3 is triggered when new medical bills are received by the health plan for members who have a subrogation file set up (and thus, a case established with the ZAF Subrogation System). Those bills are sent, along with the unique identifier associated with the member's ZAF case, by way of an HTTP Post Request through the ZAF web servers to the ZAF endpoint API. The ZAF endpoint API stores the bills in the member's case file. These bills become an important piece of evidence regarding the member's specific personal injury case. The HPC initially assumes every bill received after the creation of the subrogation file is case related. Later, this evidence will be manually sorted by ZAF to determine which bills are actually case related and which are not. In an example implementation of the ZAF Subrogation System, Query 3 is sent to api.zaflegal.com/bills.

Query 4. Query 4 is another recurring query, although it does not need to be executed as frequently as the other recurring queries (for example, every two weeks is typically sufficient). Query 4 requests a list of cases that are approaching settlement status (determined by the ZAF Subrogation System based on whether the case has reached the negotiation stage). The ZAF endpoint API responds with a list of cases that are approaching settlement. The HPC sends an automated response that includes a ledger of all medical claims paid from the date of accident to present and a sum of all claims paid for each case that is approaching settlement (in JSON format). Every time the ZAF REST API endpoint receives this query, it updates the ZAF database with the latest subrogation ledger and sum of all claims paid by the health plan. The ZAF legal team uses this information to calculate and adjust the amount of subrogation payable to the health plan. A verification procedure (e.g., human-to-human) occurs to confirm the final payoff amount of the subrogation claim. Subrogation is then paid. Payment results in the subrogation file being closed by the HPC, which terminates the query process as bills are received for the member who had a ZAF case. In an example implementation of the ZAF Subrogation System, Query 4 is sent to api.zaflegal.com/bills.

Query 5. Query 5 is another recurring query, although it does not need to be made as frequently as the other recurring queries (for example, every two weeks is typically sufficient) and in some implementations is optional. Query 5 asks the question, “Are you ready to pay off our subrogation claim?” The JSON data attached to this query is a ledger of all medical claims paid from the date of accident to present and a sum of all claims paid. Every time the ZAF endpoint API receives this query, it updates the ZAF Database with the latest subrogation ledger and sum of all claims paid by the health plan. An automated response may or may not be provided. The ZAF Subrogation System uses this information to calculate and adjust the amount payable to the health plan. A verification procedure (e.g., human-to-human) may also takes place to confirm the final payoff amount of the subrogation claim. Subrogation is paid and the ZAF Database is updated to reflect this status. This updated status creates a “no” response to any further Query 5 requests and the process ends. In an example implementation of the ZAF Subrogation System, Query 5 request is sent to api.zaflegal.com/settlement.

More specifically, FIGS. 2A-2B provide a flowchart depicting an example implementation of the disclosed subrogation system and method wherein a series of queries executed by the subrogation collection software is shown in chronological order. With direct reference to FIGS. 2A-2B, query system 200 begins at step 202. At step 204, Recurring Query 1 (e.g., DAILY) queries: are you interested in following up with this member about a potential case? Recurring Query 1 attaches member diagnoses and contact info from the health plan database at step 206. Endpoint API/Rest API (https: request API) API.zaflegal.com/suspecteddiagnosis sorted data sent to database occurs at step 208 and sorted data lands in the ZAF database at step 210. ZAF response 212 may be no (214) or yes (216). At step 220, Recurring Query 2 (e.g., DAILY) queries: what cases have you accepted/rejected/still contacting? Endpoint API or Rest API (https: Request API) API.zaflegal.com/checkleadstatus occurs at step 222 and the ZAF database is accessed at step 224. ZAF response 226 may be either these cases were rejected (228) or these cases are still being contacted (230). If the cases were accepted, Query 3 is triggered at step 232. At step 234, Query 3 queries: here is more member info for accepted cases; will you send us case information so we can set up subrogation file? Endpoint API or Rest API (https: Request API) API.zaflegal.com/subrogationfile occurs at 236 and additional member information lands in the ZAF database at 238. If the ZAF response at step 240 is yes, response 242 will contain client, date of accident, bodily injury insurer information, claim information, and case status; and a subrogation file will be created in the health plan database at step 244. At step 250, Automatic Query 4 (e.g., each time a bill is received with service date on or after date of accident) queries: here is a bill from subrogation file, should we keep sending bills? Endpoint API or Rest API (https: Request API) API.zaflegal.com/bills occurs at 252 and the ZAF database is accessed at step 254. The ZAF response at 256 may be either yes, keep sending bills (258), or no, stop sending bills for this case (260). At step 262, Recurring Query 5 (e.g. WEEKLY) queries: are you ready to reimburse the money we paid on this case (here is everything we have paid)? Endpoint API or Rest API (https: Request API) API.zaflegal.com/settlement occurs at 264; a verification process (e.g., human-to-human) to confirm final subrogation payoff occurs at step 266; and subrogation is paid at step 268.

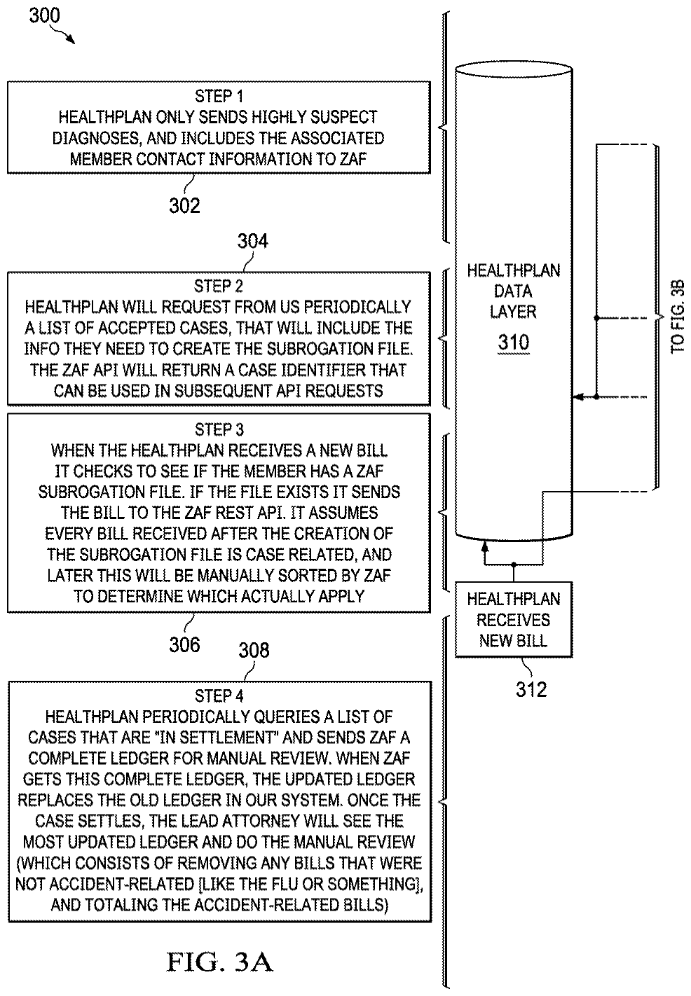

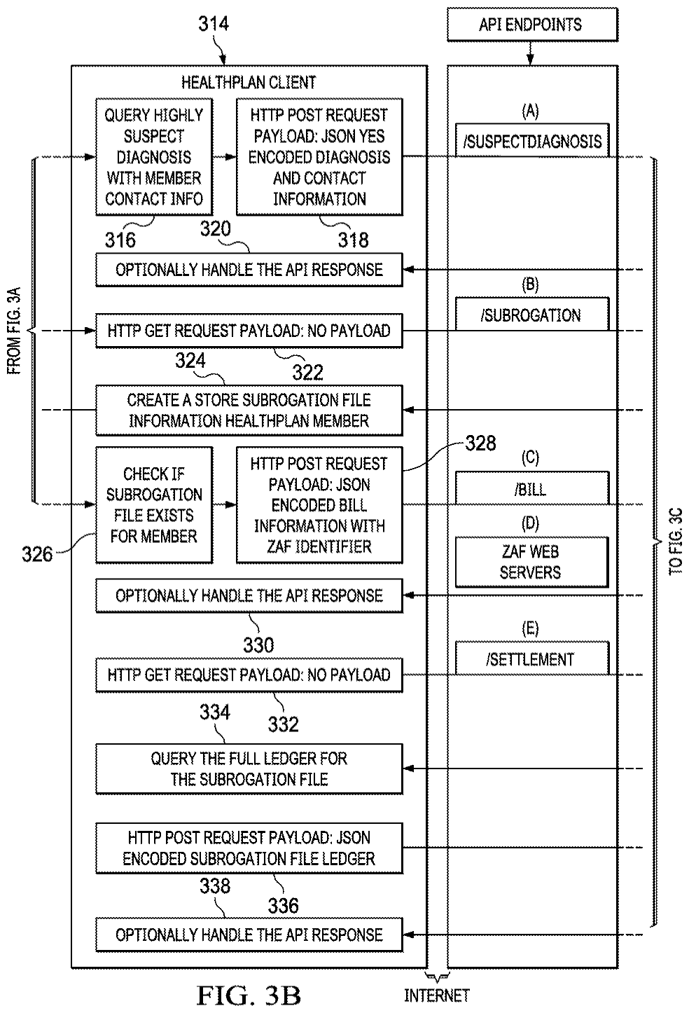

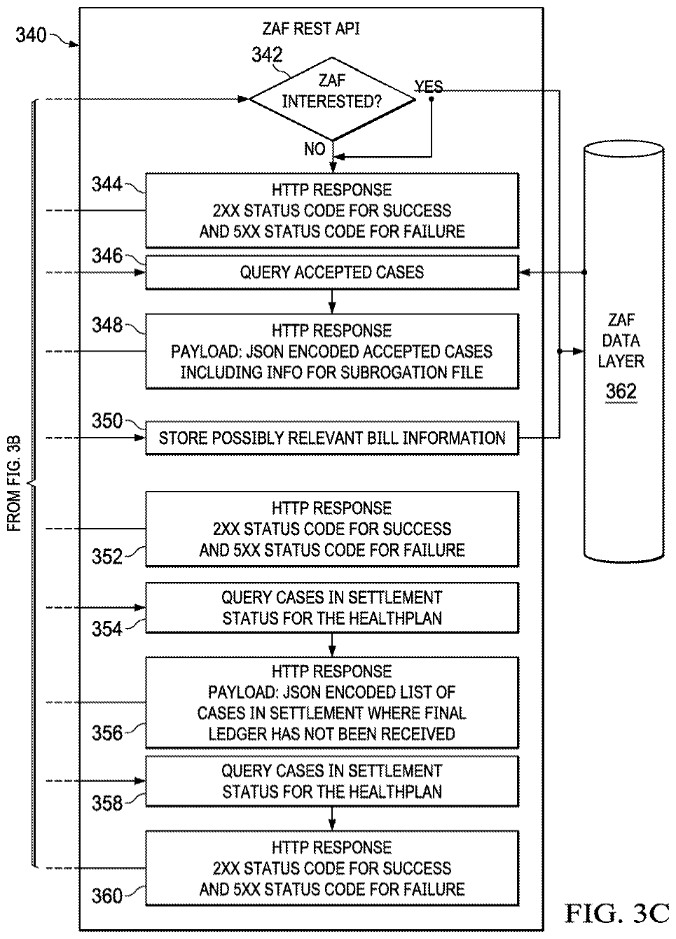

With reference generally to FIGS. 3A-3C, the case intake and the subsequent subrogation process are facilitated by certain automated data exchanges. The process depicted in FIGS. 3A-3C is a somewhat simplified iteration of the process depicted in FIGS. 2A-2B, but a more detailed technical architecture is depicted. In an example implementation, the chronological steps are as discussed below.

Step 1. In a first step, a health plan software program, or health plan client (“HPC”) interfaces with the health plan data layer and extracts highly suspect diagnoses along with associated member contact information and transmits this data payload in JSON format through an HTTP POST Request to the ZAF REST API. Predetermined criteria determines which data is stored in the ZAF Data Layer, and which members will be contacted.

Step 2. In a second step, the HPC periodically requests a list of accepted cases from ZAF Subrogation System. The HPC makes a HTTP GET Request to the ZAF REST API, which then extracts case information for all accepted cases and makes an HTTP Response with JSON formatted data that includes all the data needed by the health plan to set up an electronic subrogation file for each case accepted by ZAF Subrogation System. The HPC creates the subrogation file and stores it in the health plan data layer.

Step 3. In a third step, each time the HPC receives a new bill for any of its members, the HPC verifies that the member has an open subrogation file with ZAF Subrogation System. If a subrogation file exists, the HPC directs the bill to the ZAF REST API. The HPC assumes every bill received after the “Date of Accident” that is listed in the subrogation file is related to the personal injury case being handled by ZAF Subrogation System. Later, these bills will be categorized by ZAF Subrogation System and/or the HPC to determine what bills were and were not related to the personal injury case. When a bill is received for a member who has an open ZAF Subrogation file, it is transmitted in JSON format through an HTTP POST Request with the ZAF identifier to the ZAF REST API. This payload gets attached to the relevant case file in the ZAF data layer.

Step 4. In a fourth step, the HPC periodically queries a list of cases that are “In Settlement” and forwards a complete ledger of medical bills paid by the health plan on behalf of the member since the date of accident to ZAF Subrogation System. When ZAF Subrogation System receives this complete ledger, the updated ledger replaces the previous ledger in the ZAF Subrogation System. Once the case settles, the ZAF Subrogation System (or the lead attorney handling the case) will review the most updated ledger and extract any bills that were not accident-related (e.g., for illness), and total the accident-related bill.

More specifically, FIGS. 3A-3C provide a system schematic depicting another example implementation of the disclosed system and method for collecting subrogation in a personal injury case, wherein the various steps in the method are automatically executed by one or more servers communicating with each other using a web-based system, wherein one or more of the servers includes at least one processor for running subrogation collection software. With direct reference to FIGS. 3A-3C, at Step 1 (302), the health plan only sends highly suspect diagnoses, and includes the associated member contact information to ZAF. At Step 2 (304), the health plan periodically requests a list of accepted cases that include the information needed to create a subrogation file. The ZAF API returns a case identifier that can be used in subsequent API requests. At step 3 (306), when the health plan receives a new bill, it checks to see if the member has a ZAF subrogation file. If the file exists it sends the bill to the ZAF Rest API. It assumes every bill received after the creation of the subrogation file is case related, and later this will be manually sorted by ZAF to determine which actually apply. At step 4 (308), the health plan periodically queries a list of cases that are “in settlement” and sends ZAF a complete ledger for manual review. When ZAF receives this complete ledger, the updated ledger replaces the old ledger in the system. Once the case settles, the lead attorney will see the most updated ledger and do the manual review (which consists of removing any bills that were not accident-related [e.g., based on sickness], and totaling the accident-related bills). The system architecture includes health plan data layer 310 (at which the health plan receives a new bill at 312), health plan client 314, API endpoints, ZAF rest API, and ZAF data layer 362.

Health plan client 314 includes the following steps and aspects. Querying a highly suspect diagnosis with member contact information at 316; http post request payload: JSON Yes encoded diagnosis and contact information at 318; optionally handling the API response at 320; http get request payload: no payload at 322; creating a store subrogation file information health plan member at 324; checking in subrogation file exists for member at 326; http post request payload: JSON encoded bill information with ZAF identifier 328; optionally handling the API response at 330; http get request payload: no payload at 332; querying the full ledger for the subrogation file at 334; http post request payload: JSON encoded subrogation file ledger at 336; and optionally handling the API response at 338. The API endpoints include: (A) /suspectdiagonsis; (B) /subrogation; (C) /bill; (D) ZAF web servers; and (E) /settlement. ZAF REST API 340 includes the following includes the following steps and aspects: ZAF interested decision 342 (yes/no); http response 2xx status code for success and 5XX status code for failure 344; querying accepted cases at step 346; http response payload: JSON encoded accepted cases including info for subrogation file 348; storing possibly relevant bill information at step 350; http response 2xx status code for success and 5xx status code for failure 352; querying cases in settlement status for the health plan at step 354; http response payload: JSON encoded list of cases in settlement where final ledger has not been received 356; querying cases in settlement status for the health plan at step 358; and http response 2xx status code for success and 5xx status code for failure 360. The ZAF data layer is shown as 362 in FIG. 3C.

Advantages of the disclosed technology include the aspects discussed below, as well as other advantages. A first advantage is the manner in which the ZAF Subrogation System identifies subrogation opportunities. The disclosed technology assists health plans to identify significantly more existing personal injury cases by incentivizing health plan members to pursue their claims by using the ZAF Subrogation System for relatively low-cost or zero attorney fee legal representation. Currently, accident victims are actually incentivized to hide their claims from their health plan providers to avoid paying subrogation after receiving a settlement. By using the ZAF Subrogation System, health plan members are incentivized to be cooperative with the health plan for the sake of obtaining relatively low-cost or cost-free legal representation rather than paying one-third of the amount owed in attorney fees. A second advantage is the creation of a subrogation collection fee structure that pays for itself. By accepting and managing representation on a large number of personal injury cases, the ZAF Subrogation System generates significant subrogation revenue that health plans are currently not receiving. This additional revenue offsets all or at least part of the cost of the ZAF Subrogation System to the health plan. A third advantage is transforming an adversarial relationship into a symbiotic relationship based on data sharing between personal injury lawyers and health plans. The health plan shares data with the ZAF Subrogation System that facilitates the handling of personal injury cases such as, for example, member information needed for case intake and medical bills used to prove damages. The ZAF Subrogation System shares data that the health plan needs to assert subrogation, bridging a major information gap that currently impedes subrogation collection. A fourth advantage is encouraging more accident victims to pursue their personal injury recovery rights. Currently, many accident victims forego their claims out of fear of being perceived as greedy, vindictive, or litigious. Under the ZAF system, personal injury legal services are perceived as essentially just another health-centered plan benefit. A fifth advantage is derived from ZAF's no-cost/low-cost attorney fee structure, which substantially increases the member's net settlement. This in turn reduces litigation.

As previously stated and as used herein, the singular forms “a,” “an,” and “the,” refer to both the singular as well as plural, unless the context clearly indicates otherwise. The term “comprising” as used herein is synonymous with “including,” “containing,” or “characterized by,” and is inclusive or open-ended and does not exclude additional, unrecited elements or method steps. Although many methods and materials similar or equivalent to those described herein can be used, particular suitable methods and materials are described herein. Unless context indicates otherwise, the recitations of numerical ranges by endpoints include all numbers subsumed within that range. Furthermore, references to “one implementation” are not intended to be interpreted as excluding the existence of additional implementations that also incorporate the recited features. Moreover, unless explicitly stated to the contrary, implementations “comprising” or “having” an element or a plurality of elements having a particular property may include additional elements whether or not they have that property.

Underlined and/or italicized headings and subheadings are used for convenience only, do not limit the disclosed subject matter, and are not referred to in connection with the interpretation of the description of the disclosed subject matter. All structural and functional equivalents to the elements of the various implementations described throughout this disclosure that are known or later come to be known to those of ordinary skill in the art are expressly incorporated herein by reference and intended to be encompassed by the disclosed subject matter. Moreover, nothing disclosed herein is intended to be dedicated to the public regardless of whether such disclosure is explicitly recited in the above description.

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted by any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

It should be appreciated that all combinations of the foregoing concepts and additional concepts discussed in greater detail herein (provided such concepts are not mutually inconsistent) are contemplated as being part of the disclosed technology. In particular, all combinations of claimed subject matter appearing at the end of this disclosure are contemplated as being part of the technology disclosed herein. While the disclosed technology has been illustrated by the description of example implementations, and while the example implementations have been described in certain detail, there is no intention to restrict or in any way limit the scope of the appended claims to such detail. Additional advantages and modifications will readily appear to those skilled in the art. Therefore, the disclosed technology in its broader aspects is not limited to any of the specific details, representative devices and methods, and/or illustrative examples shown and described. Accordingly, departures may be made from such details without departing from the spirit or scope of the general inventive concept.

What is claimed:

1. A system for collecting subrogation from personal injury settlements, comprising:
(a) a computer network, wherein the computer network includes servers, processors, databases, and software;
(b) wherein the computer network is configured to facilitate establishment of a contractual relationship between a health plan provider and a legal entity acting as a subrogation collection entity;
(c) wherein the computer network is configured to facilitate establishment of a contractual relationship between the health plan provider and a constituent of a health plan offered by the health plan provider, wherein the health plan includes access by the constituent of the health plan to personal injury legal services offered by the legal entity acting as a subrogation collection entity at either no cost or reduced cost to the constituent of the health plan;
(d) wherein the computer network is configured to access information stored in one or more databases that includes electronic health information about the constituent of the health plan;
(e) wherein the stored information includes medical diagnosis and procedure codes indicative of an accident in which the constituent of the health plan has been involved that results in a personal injury case is periodically reviewed;
(f) wherein the computer network is configured to contact the constituent of the health plan and gains approval from the constituent of the health plan to utilize the legal services of the subrogation collection entity;
(g) wherein the computer network is configured to create a subrogation case file for the health plan upon receiving approval from the constituent to utilize the personal injury legal services of the legal entity acting as a subrogation collection entity;
(h) wherein the computer network is configured to retrieve information from the databases that includes medical records of the constituent of the health plan regarding accident-related medical expenses paid to the constituent of the health plan by the health plan provider and use the accident-related medical expenses to calculate total subrogation payable to the health plan provider;
(i) wherein the computer network is configured to obtain approval from the constituent of the health plan to facilitate personal injury legal services and settle the personal injury case of the constituent of the health plan at no cost or reduced cost to the constituent of the health plan; and
(j) wherein the computer network is configured to facilitate payment of up to 100% of the calculated subrogation by the subrogation collection entity to the health plan provider.

2. The system of claim 1, wherein the system charges a predetermined fee to the health plan provider for software and services of the legal entity acting as a subrogation collection entity.

3. The system of claim 1, wherein the legal entity acting as a subrogation collection entity has access to the databases and servers of a personal injury lawyer or personal injury law firm.

4. The system of claim 1, wherein the system is entirely automated.

5. The system of claim 1, wherein predetermined aspects of the system are automated.

6. The system of claim 1, wherein the computer network may be accessed using application software resident on hand-held devices or by a browser-based web applications accessible on any device having an internet connection.

7. The system of claim 6, wherein the hand-held devices include mobile telephones and tablet computers.

8. A computerized method for collecting subrogation in a personal injury case, comprising:
(a) using a computer network that includes servers, processors, databases, and software to facilitate the establishment of a contractual relationship between a health plan provider and a legal entity acting as a subrogation collection entity;
(b) using the computer network to facilitate the establishment of a contractual relationship between the health plan provider and a constituent of a health plan offered by the health plan provider, wherein the health plan includes access by the constituent of the health plan to personal injury legal services offered by the legal entity acting as a subrogation collection entity at no cost or reduced low cost to the constituent of the health plan;
(c) using the computer network to access information stored in one or more databases that includes electronic health information about the constituent of the health plan;
(d) periodically reviewing the stored information includes medical diagnosis and procedure codes indicative of an accident in which the constituent of the health plan has been involved, wherein the accident results in a personal injury case;
(e) using the computer network to contact the constituent of the health plan and gain approval from the constituent of the health plan to utilize the legal services of the subrogation collection entity;
(f) using the computer network to create a subrogation case file for the health plan upon receiving approval from the constituent to utilize the personal injury legal services of the legal entity acting as a subrogation collection entity;
(g) using the computer network to retrieve information from the databases that includes medical records of the constituent of the health plan regarding accident-related medical expenses paid to the constituent of the health plan by the health plan provider and using the accident-related medical expenses to calculate total subrogation payable to the health plan provider;
(h) using the computer network to obtain approval from the constituent of the health plan to settle the personal injury case of the constituent of the health plan at no cost or reduced cost to the constituent of the health plan; and
(i) using the computer network to facilitate payment of up to 100% of the calculated subrogation by the subrogation collection entity to the health plan provider.

9. The method of claim 8, further comprising charging a predetermined fee to the health plan provider for software and services of the legal entity acting as a subrogation collection entity.

10. The method of claim 8, wherein the legal entity acting as a subrogation collection entity has access to the databases and servers of a personal injury lawyer or personal injury law firm.

11. The method of claim 8, wherein the method is entirely automated.

12. The method of claim 8, wherein predetermined aspects of the method are automated.

13. The method of claim 8, wherein the computer network may be accessed using application software resident on hand-held devices or by a browser-based web applications accessible on any device having an internet connection.

14. The method of claim 8, wherein the hand-held devices include mobile telephones and tablet computers.

15. An automated method for collecting subrogation in a personal injury case, comprising:
(a) providing a computer network, wherein the computer network includes servers, processors, databases, and software;
(b) using the computer network to facilitate the establishment of a contractual relationship between a health plan provider and a legal entity acting as a subrogation collection entity;
(c) using the computer network to facilitate the establishment of a contractual relationship between the health plan provider and a constituent of a health plan offered by the health plan provider, wherein the health plan includes access by the constituent of the health plan to personal injury legal services offered by the legal entity acting as a subrogation collection entity at no cost or reduced low cost to the constituent of the health plan;
(d) using the computer network to access information stored in one or more databases that includes electronic health information about the constituent of the health plan;
(e) periodically reviewing the stored information includes medical diagnosis and procedure codes indicative of an accident in which the constituent of the health plan has been involved, wherein the accident results in a personal injury case;
(f) using the computer network to contact the constituent of the health plan and gain approval from the constituent of the health plan to utilize the legal services of the subrogation collection entity;
(g) using the computer network to create a subrogation case file for the health plan upon receiving approval from the constituent to utilize the personal injury legal services of the legal entity acting as a subrogation collection entity;
(h) using the computer network to retrieve information from the databases that includes medical records of the constituent of the health plan regarding accident-related medical expenses paid to the constituent of the health plan by the health plan provider and using the accident-related medical expenses to calculate total subrogation payable to the health plan provider;
(i) using the computer network to obtain approval from the constituent of the health plan to settle the personal injury case of the constituent of the health plan at no cost or reduced cost to the constituent of the health plan;
(j) using the computer network to facilitate payment of up to 100% of the calculated subrogation by the subrogation collection entity to the health plan provider; and
(k) charging a predetermined fee to the health plan provider for the software and services of the legal entity acting as a subrogation collection entity.

16. The method of claim 15, wherein the legal entity acting as a subrogation collection entity has access to the databases and servers of a personal injury lawyer or personal injury law firm.

17. The method of claim 15, wherein the computer network may be accessed using application software resident on hand-held devices or by a browser-based web applications accessible on any device having an internet connection.

18. The method of claim 17 wherein the hand-held devices include mobile telephones and tablet computers.
